(19) United States
(12) Patent Application Publication
(10) Pub. No.: US 2022/0209577 A1
(43) Pub. Date: Jun. 30, 2022

(54) SYSTEM AND METHOD FOR HEAT MITIGATION OF AN IMPLANTABLE MEDICAL DEVICE DURING WIRELESS CHARGING

(71) Applicant: Advanced Neuromodulation Systems Inc., Plano, TX (US)

(72) Inventors: Luis Ortiz Hernandez, Plano, TX (US); Robert Nobles, Plano, TX (US); Santhosh Seetharam, Plano, TX (US); Thomas Daigneault, Plano, TX (US)

(21) Appl. No.: 17/464,590

(22) Filed: Sep. 1, 2021

Related U.S. Application Data
(60) Provisional application No. 63/132,353, filed on Dec. 30, 2020.

(51) Int. Cl. H02J 50/10; H02J 7/00; A61N 1/378

(52) U.S. Cl. CPC H02J 50/10; H02J 7/007192; A61N 1/3787; H02J 7/00712; H02J 7/00032; H02J 7/0047

(57) ABSTRACT

An Implantable Medical Device (IMD) communicates to a charger device using an RF communications channel. Temperature sensors in the IMD obtain temperature measurements during wireless charging, and the IMD then transmits the temperature measurements to the charger device over the RF communications channel. Using these temperature measurements, the charger device determines whether heat mitigation for the IMD is needed during wireless charging.

### CROSS REFERENCE TO RELATED APPLICATIONS

This application claims priority under 35 U.S.C. § 119 to U.S. Provisional Application No. 63/132,353 entitled, “SYSTEM AND METHOD FOR HEAT MITIGATION OF AN IMPLANTABLE MEDICAL DEVICE DURING WIRELESS CHARGING,” filed Dec. 30, 2020, and hereby expressly incorporated by reference herein.

### FIELD

The present disclosure relates generally to an implantable medical device (IMD) with a rechargeable battery and more particularly, to a system and method for mitigation of heat in the IMD during wireless charging of the rechargeable battery.

### BACKGROUND

The statements in this section provide a description of related art and are not admissions of prior art. No admission is made that the related art described herein is publicly available or known to others besides the inventors.

An implantable medical device (IMD) is partially or totally introduced, surgically or medically, into the body of a patient, human or non-human and typically includes one or more electrodes that deliver electrostimulation to tissue for diagnostic or therapeutic purposes. An IMD may include a neurostimulation device configured for spinal cord stimulation, deep brain stimulation, cortical stimulation, cochlear nerve stimulation, peripheral nerve stimulation, vagal nerve stimulation, sacral nerve stimulation, and others. In the example of a neurostimulation device for spinal cord stimulator (SCS), the IMD is configured to treat chronic pain by delivering stimulation pulses to a patient's spinal cord that induces paresthesia in regions of the patient's body. Other examples of IMDs may include pacemakers for treating cardiac arrhythmia, defibrillators for treating cardiac fibrillation, cochlear stimulators for treating deafness, retinal stimulators for treating blindness, or muscle stimulators for producing coordinated limb movement or reducing tremors. These examples are not limiting and the IMDs described herein may include any other device configured for implantation in a patient.

An IMD implanted in a patient needs a reliable power source. Some electrically operated IMDs are powered by a primary cell (commonly referred to as a non-rechargeable battery). When the battery of such an IMD is depleted, the device must be removed from the patient's body such that its battery can be replaced or a new IMD with a new battery may be implanted. To avoid removal of an IMD for battery replacement, other electrically operated IMDs include secondary cells (commonly referred to as rechargeable batteries). The rechargeable battery of such an IMD is recharged using a non-implanted or external wireless charger device. The external charger device includes an inductive coil that enables power to be wirelessly transferred, through the patient's epidermis, from the charger device to an inductive coil in the IMD to charge the rechargeable battery.

In general, to effectively recharge the IMD, the external charger device needs to be positioned over the epidermis of the patient and within a certain range and alignment of the IMD. One of the biggest challenges of wirelessly charging an IPG is the unwanted heat generated during charging. Wireless charging generates eddy currents on metal components in the charging path. Without an effective heat dissipation path, the eddy currents may accumulate heat locally and become a source of unwanted heat. This heat can cause effects from a slightly uncomfortable sensation to severe tissue damage.

Thus, there is a need for an improved system and method for mitigation of heat in an IMD during wirelessly charging. Other advantages of embodiments of the systems and methods are described herein or are apparent from implementations thereof.

### SUMMARY

The following presents a summary of the disclosed subject matter in order to present some aspects of the disclosed subject matter.

In one aspect, an external charger device includes a charging module with at least one primary coil configured to wirelessly transfer power to a charging coil in an implantable medical device (IMD). A transceiver is configured to communicate with the IMD using an RF communications channel and receive one or more temperature measurements from the IMD over the RF communications channel. At least one processing circuit and at least one memory device, wherein the at least one memory device stores instructions that, when executed by the at least one processing circuit, causes the external charger device to compare the one or more temperature measurements from the IMD to at least one heating threshold and determine to perform heat mitigation for the IMD when the one or more temperature measurements exceed the at least one heating threshold.

In a second aspect, an external device includes a transceiver configured to communicate with an implantable medical device (IMD) using an RF communications channel. The external device further includes at least one processing device and at least one memory device, wherein the at least one memory device stores instructions that, when executed by the at least one processing device, causes the external device to obtain at least one temperature measurement from the IMD; determine a temperature slope using the at least one temperature measurement and a charging time; compare the temperature slope to a heating threshold; and when the temperature slope exceeds the heating threshold, determine to lower a power output of an external charger device.

In a third aspect, a method includes initiating wireless charging of an implantable medical device (IMD); receiving one or more temperature measurements from the IMD over an RF communications channel; comparing the one or more temperature measurements from the IMD to at least one heating threshold; and performing heat mitigation of the IMD when the one or more temperature measurements exceed the at least one heating threshold.

In one or more of the above aspects, the external charger device is configured to perform the heat mitigation for the IMD by adjusting a power output of the charging module.

In one or more of the above aspects, the at least one heating threshold includes a predetermined temperature after a predetermined time period of wireless charging.

In one or more of the above aspects, the external charger device is configured to process an input to lower an operating temperature of the IMD; decrease a power output of the charging module; and adjust the at least one heating threshold in response to the input. In one or more of the above aspects, the external charger device is configured to monitor a power output range of the charging module and compare the power output range to one or more power thresholds. The external charger device is further configured to adjust a power output of the charging module when the power output range exceeds at least one of the power thresholds.

In one or more of the above aspects, the external charger device is configured to adjust a power output of the charging module when the power output range exceeds the at least one of the power thresholds and when the one or more temperature measurements exceed the at least one temperature threshold.

In one or more of the above aspects, the external charger device is further configured to monitor a plurality of charging parameters of the charging module, wherein the one or more charging parameters include one or more of: a power output, a bridge current, a bridge voltage, or a phase difference between the bridge current and the bridge voltage and compare the plurality of charging parameters to corresponding one or more charging thresholds.

In one or more of the above aspects, an external device is configured to process an input originated from a user to lower an operating temperature of the IMD and adjust the heating threshold in response to the input.

In one or more of the above aspects, the external device is configured to adjust a power output of the external charger device when the one or more temperature measurements exceed the at least one heating threshold.

In one or more of the above aspects, the external device may include one or more of a patient controller or a charger device.

Additional aspects are set forth, in part, in the detailed description, figures and claims which follow, and in part may be derived from the detailed description, or may be understood by practice of the embodiments. It is to be understood that the description herein is exemplary and explanatory only and is not restrictive of the embodiments as claimed.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings may indicate similar, equivalent, or identical components or a different embodiment of a component.

FIG. 1 is a schematic block diagram illustrating an embodiment of selected components of an implantable medical device (IMD) and external devices.

FIG. 2 is a schematic block diagram of an embodiment of a system illustrating an RF communication channel between an IMD and a charger device.

FIG. 3 is a schematic block diagram of an embodiment of a method illustrating events that may trigger communication messages between the IMD and the charger device over an RF communications channel and/or a near field communications channel.

FIG. 4 is a logical flow diagram of an embodiment of a method for monitoring temperatures of an IMD 100 by a charger device.

FIG. 5 is a logical flow diagram of an embodiment of a method for heat mitigation of an IMD.

FIG. 6 is a logical flow diagram of another embodiment of a method for heat mitigation of an IMD.

FIG. 7 is a logical flow diagram of another embodiment of a method for determining to perform heat mitigation for an IMD.

FIG. 8 is a graphical representation of an exemplary correlation between power output of a charger device and a temperature of an IMD 100 after 15 minutes of charging.

FIG. 9 is a logical flow diagram of an embodiment of a method for modifying power output of a charger device.

FIG. 10 is a schematic block diagram of an embodiment of a graphical user interface (GUI) for power management of a charger device.

FIG. 11 is a schematic block diagram of an embodiment of an exemplary network in which a charger device and a patient controller may operate.

### DETAILED DESCRIPTION

The description and drawings merely illustrate the principles of various embodiments. Additional arrangements, although not explicitly described or shown herein, are intended to be included within a scope of the disclosure. Furthermore, examples recited herein are intended for pedagogical purposes to aid in understanding the principles of the embodiments and are not intended to limit the scope to such specifically recited examples. Moreover, statements herein reciting principles, aspects, and embodiments, as well as specific examples thereof, are intended to encompass equivalents thereof.

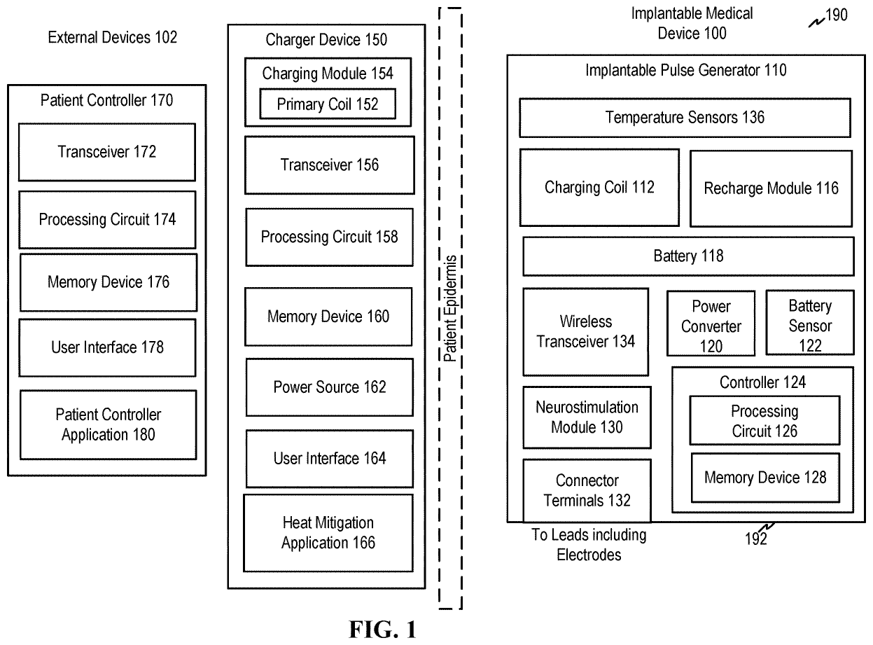

FIG. 1 is a schematic block diagram illustrating an embodiment of selected components of an implantable medical device (IMD) 100 and exemplary external devices 102. The system 190 in FIG. 1 is intended to be exemplary, and in other implementations may include additional or alternative components or devices. The patient referred to herein may be any user, human or non-human, of the IMD 100.

The IMD 100 shown in FIG. 1 is typically implanted under an epidermal layer internally within tissue of the patient. The IMD 100 is generally implanted subcutaneously at depths ranging, e.g., from 5 mm to 25 mm depending upon the application and patient. In other embodiments not shown, the IMD 100 may be wholly or partially external to the patient, such as adjacent to or partially implanted within the epidermal layer and tissue of the patient. In the example of FIG. 1, the IMD 100 is an implantable pulse generator (IPG) 110 configured for spinal cord stimulation or deep brain stimulation, though a person of skill in the art will understand that other types of IMDs 100 may also implement one or more of the embodiments described herein.

The IPG 110 includes a charging coil 112, a recharge module 116, battery 118, power converter 120 and battery sensor 122. The battery 118 is a rechargeable battery such as a lithium ion battery, but is not limited thereto. The recharge module 116 is operable to wirelessly receive externally generated power through the charging coil 112, and use the externally generated power to charge the battery 118. The power converter 120 converts power from the battery 118 for transfer to one or more components of the IPG 110. The battery sensor 122 determines a power level of the battery 118 and provides alerts, e.g., when the battery 118 is fully charged or when the battery 118 is low on power.

A controller 124 includes at least one processing circuit 126 and at least one memory device 128 and is configured to control one or more functions of the IPG 110 described herein. The memory device 128 is a non-transitory, processor readable medium that stores programs, code, states, instructions and/or data which when executed or processed by the processing circuit 126, causes the IPG 110 to perform one or more functions described herein.

The IPG 110 further includes a neurostimulation module 130 configured to generate electrical pulses for delivery by electrodes to target neural tissue. The IPG 110 is coupled to the electrodes via one or more leads (not shown). The connector terminals 132 couple the leads to the IPG 110. The neurostimulation module 130 delivers electrical pulses in accordance with selected neurostimulation parameters, which can specify a lead, an electrode configuration for the specified lead, and one or more pulse parameters, including, but not limited to, pulse amplitude, pulse width and pulse repetition rate parameters.

In an embodiment, the IPG 110 communicates with a charger device 150 using near field communication, such as reflected impedance modulation, which is sometimes known in the art as Load Shift Keying (LSK) or Amplitude-shift keying (ASK). LSK, which is a particular form of ASK, is a communication scheme which allows simultaneous powering and data transmission through inductive coupling, e.g. of the charging coil 112 with a primary coil 152 of the external charger device 150. A change of the load on the charging coil 112 is reflected onto the primary coil 152 as a varying impedance (i.e., reflected impedance). A near field communication protocol is used to communicate information to the charger device 150 during charging. For example, the IPG 110 communicates that charging is initiated, the battery 118 is fully charged, or charging has halted.

In this embodiment, a wireless transceiver 134 in the IPG 110 is configured to communicate with a patient controller 170 using a proprietary wireless RF communication protocol or a standard wireless RF communication protocol, e.g. such as the wireless Bluetooth™ protocol standard. The wireless transceiver 134 may additionally or alternatively use another wireless RF communication protocol with the patient controller 170, e.g. such as the Medical Implant Communication Service (MICS) standard, which was defined by the U.S. Federal Communications Commission (FCC) and European Telecommunications Standards Institute (ETSI). The MICS standard uses the RF band between 402 and 405 MHz to provide for bi-directional radio communication with implantable medical devices (IMDs), such as the IPG 110. In 2009 the FCC began referring to the RF band between 402 and 405 MHz as being part of the 401 to 406 MHz Medical Device Radio communications (MedRadio) Service band. Accordingly, the RF band between 402 and 405 MHz can be referred to as the MICS/MedRadio band, and the communication standards relating to the MICS/MedRadio band can be referred to as the MICS/MedRadio communication standards. Alternatively, the wireless transceiver 134 can perform wireless RF communications with the patient controller 170 using the Industrial, Scientific, and Medical (ISM) radio bands. The IPG 110 may also perform wireless communication with the patient controller 170 using the 3GPP Release 13, eMTC, NB-IOT or EC-GSM-IoT standards, and in particular the Internet of Medical Things (IoMT) applications of such standards. The use of other standards and frequency bands are also possible.

The IPG 110 typically includes at least one printed circuit board (PCB) with the above various electronic components mounted thereto. The at least one PCB may include the charging coil 112 as well as a second coil for use as an antenna for the wireless transceiver 134. In another embodiment, the charging coil 112 may be wrapped around the PCB within a housing 192 of the IPG 110. The various components on the PCB may be coupled directly or indirectly via separate buses or via a shared data bus.

The external devices 102 are non-implanted or non-implantable devices and are external to the epidermal layer of the patient. The external devices 102 in this example of system 190 include a charger device 150 and a patient controller 170. The patient controller 170 may be a dedicated control device or a non-dedicated user device, such as a smart phone, smart tablet, smart watch, laptop, desktop, or any other external control device configured to control the IPG 110. The patient controller 170 includes a transceiver 172 that is configured to communicate at least with the wireless transceiver 134 of the IPG 110 and with the charger device 150, using one or more wireless communication protocols, e.g. such as described herein with respect to the wireless transceiver 134 of the IPG 110.

The patient controller further includes a processing circuit 174, memory device 176, and user interface 178. The user interface 178 may include one or more of a display, keyboard, touchscreen, touchpad, mouse or other such input or output devices. The memory device 176 is a non-transitory, processor readable medium that stores programs, code, states, instructions and/or data which when executed or processed by the processing circuit 174, causes the patient controller 170 to perform one or more functions described herein.

A patient controller application 180 is configured to adjust parameters of the IPG 110 in accordance with a patient's prescribed medical program. For example, the patient may control a mode of the IPG 110 (Airplane Ready Mode, Surgery Mode or MRI Mode) or a type of therapy program (continuous, intermittent or sleep) or a strength of the stimulation pulses. The patient controller 170 receives the control commands from the patient through the user interface 178 and transmits the control commands to the IPG 110. The patient controller 180 may also receive data from the IPG 110 and provide information about the operation of the IPG 110 to the patient through the user interface 178.

The charger device 150 includes a transceiver 156, processing circuit 158, memory device 160, power source 162 and user interface 164. The transceiver 156 is configured to communicate with the transceiver 172 of the patient controller, e.g. such as described hereinabove. The memory device 160 is a non-transitory, processor readable medium that stores programs, code, states, instructions and/or data which when executed or processed by the processing circuit 158 enables the charger device 150 to perform one or more functions described herein. The user interface 164 includes one or more of a display, keyboard, touchscreen, touchpad, mouse or other such input or output devices. The user interface 164 allows a patient or clinician to input commands to the charger device 150 and receive information from the charger device 150.

The charger device 150 further includes a charging module 154 including a primary coil 152 configured for power transmission to the charging coil 112 of the IPG 110. Power transmission from the charger device 150 to the IPG 110 occurs wirelessly and transcutaneously through the patient's epidermis and tissue, via inductive coupling. Such an inductive coupling enables the IPG 110 to wirelessly receive power from the charger device 150 and recharge its battery 118. More specifically, an alternating current (AC) in the primary coil 152 generates a magnetic field with a fluctuating magnetic field strength. This fluctuating magnetic field in turn induces an AC current in the charging coil 112. The AC current is rectified and smoothed by the recharge module 116 to output a substantially constant DC voltage signal. This substantially constant DC voltage signal is then applied to charge or recharge the battery 118.

In an embodiment, as described above, the charger device 150 may communicate with the IPG 110 through inductive coupling, e.g. of the primary coil 152 of the external charger device 150 with the charging coil 112 of the IPG 110. The charger device 150 and IPG 110 may use a near field communication protocol, such as the Wireless Power Consortium (WPC) Qi wireless charging standard, Version 1.2.4 released in 2017 or other standard or proprietary protocol for near field communication during charging.

In an embodiment, the IPG 110 is configured to communicate to the charger device 150 using the charging coil and a near field communications protocol as described above and also using an RF communications channel between the wireless transceiver 134 of the IPG 110 and the transceiver 156 of the charger device 150. To assist in maintaining a predetermined temperature range of the IPG 110 during wireless charging, one or more temperature sensors 136 obtain temperature measurements of the IPG 110. The temperature sensors 136 may measure one or more of: an internal temperature, a temperature of the housing 192 of the IPG 110, or an external temperature of surrounding tissue. The IPG 110 then transmits the temperature measurements to the charger device 150. The charger device 150 includes a heat mitigation application that receives the temperature measurements from the IPG 110. The heating mitigation application 166 then determines whether heat mitigation measures are needed during wireless charging.

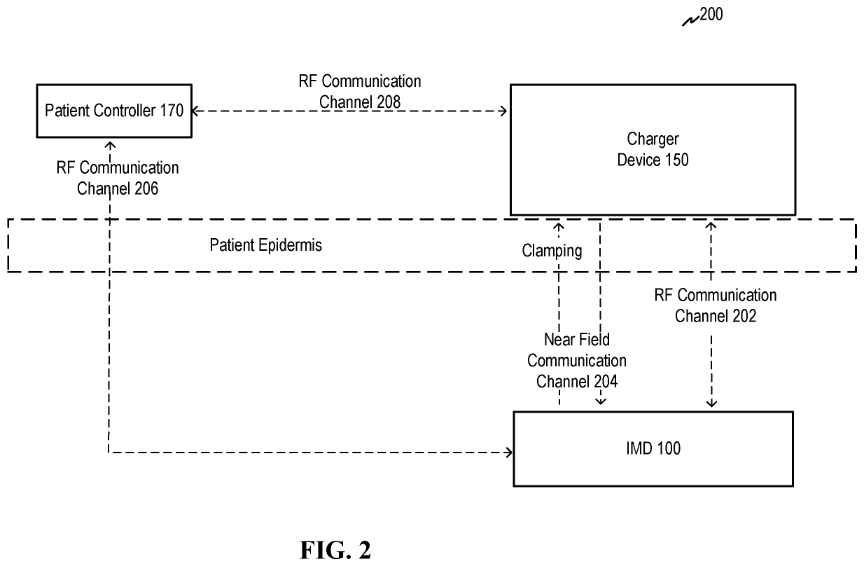

FIG. 2 is a schematic block diagram of an embodiment of a system 200 illustrating the additional RF communication channel 202 between the IMD 100 and the charger device 150. When charging the IMD 100, the housing of the charger device 150 may directly touch the patient's epidermis or in other examples, a charger holding device or the patient's clothing or both may lay between the charger device 150 and the patient's epidermis. A user moves the charger device 150 across the patient's epidermis to position the charger device 150 above the tissue under which the IMD 100 is implanted. For an efficient inductive coupling, the primary coil 152 and the charging coil 112 should be in alignment with respect to one another, e.g. the primary coil 152 and the charging coil 112 should be within a predetermined distance and have a predetermined position relative to each other. Misalignment of the charger device 150 may introduce unexpected noise, trigger false detection of a presence of the IMD 100, or start false charging. The improper positioning of the charger device 150 may also lead to inefficient charging time, high charging power consumption and/or generation of heat on undesired metal surfaces of the IMD 100.

In current legacy systems, the IMD 100 and charger device 150 communicate using a near field communication channel 204 through the primary coil 152 and the charging coil 112. The near field communication is currently limited to charging status data, such as a clamping signal from the IMD 100 to the charger device 150 to signal initiation of power transfer and charging. In general, the near field communication channel 204 is subject to noise and low data rates. The type of messages and data transmitted over the magnetic communication channel 204 is thus limited.

In an embodiment, the wireless transceiver 134 of the IMD 100 may additionally communicate with the transceiver 156 of the charger device 150 using the RF communication channel 202. For example, the IMD 100 may communicate with the charger device 150 using a proprietary wireless RF communication protocol or a standard wireless RF communication protocol, e.g. such as the wireless Bluetooth™ protocol standard. The wireless transceiver 134 of the IMD 100 may additionally or alternatively use a wireless far field communication protocol with the charger device 150, e.g. such as the Medical Implant Communication Service (MICS) standard, which was defined by the U.S. Federal Communications Commission (FCC) and European Telecommunications Standards Institute (ETSI). The MICS standard uses the RF band between 402 and 405 MHz to provide for bi-directional radio communication with implantable medical devices (IMDs), such as the IPG 110. In 2009 the FCC began referring to the RF band between 402 and 405 MHz as being part of the 401 to 406 MHz Medical Device Radio communications (MedRadio) Service band. Accordingly, the RF band between 402 and 405 MHz can be referred to as the MICS/MedRadio band, and the communication standards relating to the MICS/MedRadio band can be referred to as the MICS/MedRadio communication standards. Alternatively, the wireless transceiver 134 may perform wireless RF communications with the wireless charger 150 using the Industrial, Scientific, and Medical (ISM) radio bands. The IMD 100 may also perform wireless communication with the charger device 150 using the 3GPP Release 13, eMTC, NB-IOT or EC-GSM-IoT standards, and in particular the Internet of Medical Things (IoMT) applications of such standards. Other communication protocols and/or RF frequency bands may also be implemented by the IMD 100 and the charger device 150.

In addition, the IMD 100 communicates directly with the patient controller 170 over an RF Communication Channel 206. The patient controller 170 may transmit control commands to the IMD 100 over the RF Communication Channel 206. The patient controller 170 may also receive data from the IMD 100 over the RF Communication Channel 206 and provide information about the operation of the IMD 100 to the patient. The patient controller 170 may also communicate over an RF Communication Channel 208 to the charger device 150.

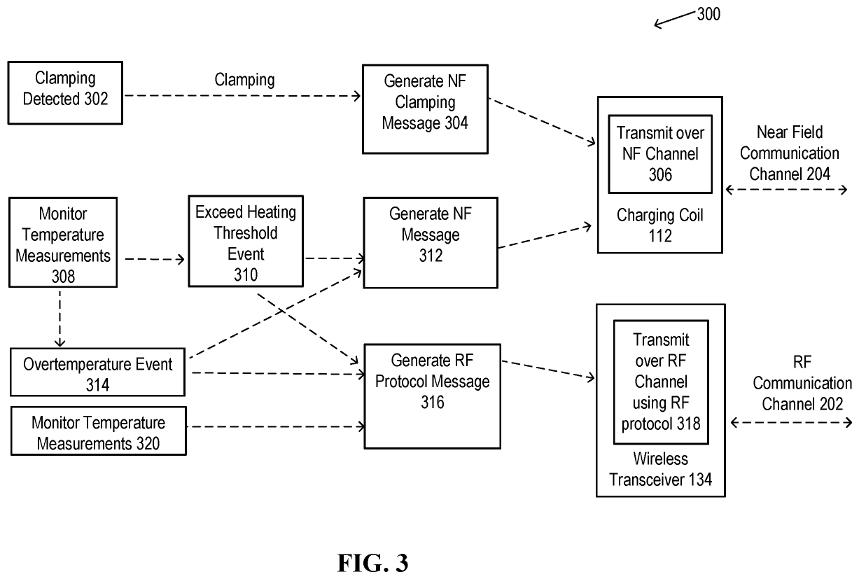

FIG. 3 is a schematic block diagram of an embodiment 300 of events that may trigger communication messages between the IMD 100 and the charger device 150 over the RF communications channel 202 and/or near field communications channel 204. When charging is initiated in the charging coil 112 of the IMD 100, a signal is generated by the IMD 100 in response to detection of clamping at 302. A near field (NF) message is generated at 304 to indicate clamping by the IMD. The NF message is then transmitted by the IMD 100 over the near field (NF) communication channel at 306 to the charger device 150. The NF message indicates to the charger device 150 that charging of the IMD 100 is initiated.

During charging, the IMD 100 monitors one or more temperature sensors 136 at 308. The temperature sensors 136 measure one or more of internal temperatures of the IMD 100, housing temperatures of the IMD 100 or temperatures of surrounding tissue. The IMD 100 compares one or more temperature measurements from the temperature sensors 136 to one or more heating thresholds. Since tissue damage depends on the temperature and the exposure time, the heating thresholds may include a temperature range and corresponding exposures times. For example, CEM43 is an industry accepted thermal dose parameter that may be implemented as at least one heating threshold herein. CEM43 includes a normalizing method to convert various time-temperature exposures applied into an equivalent exposure time expressed as minutes. Cumulative equivalent minutes at 43° C. (CEM43) is the accepted metric for thermal dose assessment that correlates well with thermal damage in a variety of tissues. The calculation of CEM43 is performed as follows:

CEM43=ΔtR(43−T)

wherein Δt signifies summation over a length of exposure, T is the average temperature during time interval t, and R is a constant equal to 0.25 for T<43° C. and 0.5 for T>43° C. The values of CEM43 have been found to correlate with severity of thermal damage. For example, CEM43 includes a first threshold of 43° C. after 30 minutes of wireless charging and a second threshold of 44° C. after 15 minutes. Thus, CEM43 may be used to define one or more of the heating thresholds. The heating thresholds may thus be a predetermined temperature at a predetermined charging time. The heating threshold may also be expressed as a temperature slope, e.g. a temperature increase per a unit of time. For example, the temperature slope may include a range of 1.4° C.-3° C. per minute.

Though CEM43 may be implemented to determine one or more of the heating thresholds for preventing tissue damage, other heating thresholds may be implemented as well. For example, the heating thresholds may be adjusted based on input from a patient, e.g. if the patient is feeling uncomfortable, the patient may generate an alert or a request to lower an operating temperature of the IMD. The user interface 178 of the patient controller 170 and/or the user interface 164 of the charger device 150 may receive the alert or request by the patient. In response thereto, one or more of the heating thresholds may be dropped by a predetermined amount (e.g., 0.1° C. to 1° C.). For example, a heating threshold may be adjusted by 0.5° C. after a first request, e.g. to 42.5° C. after 30 minutes of charging or 43.5° C. after 15 minutes of charging. In other words, the heating threshold may be established or selected to prevent tissue from being heated to an elevated level and duration that could be uncomfortable or undesirable to the patient. The heating threshold may be preset by the manufacturer and/or selected by a clinician.

When clamping is initiated, the IMD 100 begins to track a time of wireless charging. The IMD also monitors the temperature measurements from the one or more sensors 136. When one or more of the temperature measurements after a predetermined time period of wireless charging exceeds one or more heating thresholds, an “exceed heating threshold” event is triggered at 310. For example, a first heating threshold may include CEM43 of 44° C. after 15 minutes. If a temperature measurement is 44° C. after 15 minutes of charging time, the “exceed heating threshold” event is triggered at 310.

A near field (NF) message is generated by the IMD 100 to signal the “exceed heating threshold” event at 312. The NF message is then transmitted by the charging coil 112 of the IMD 100 over the near field communication channel to the charger device 150 at 306. Due to the lack of bandwidth or low signal to noise ratio, the NF message may only signal the event without further data of the temperature measurement or heating threshold.

In addition or alternatively, a radio frequency (RF) protocol message is generated in response to the “exceed heating threshold” event at 316. For example, the RF protocol message may be a Bluetooth or other wireless protocol message. The RF protocol message may indicate the “exceed heating threshold” event and also include data associated the event, such as one or more temperature measurements and one or more exceeded heating thresholds. The RF protocol message is then transmitted by the wireless transceiver 134 of the IMD 100 over the RF communications channel 202 to the charger device 150 using the RF protocol (such as Bluetooth protocol) at 318.

In another example, when one or more of the temperature measurements exceed one or more predetermined temperatures, an “overtemperature” event is triggered at 314. Another radio frequency (RF) protocol message may then be generated that indicates the “overtemperature” event at 316. The RF protocol message may not only indicate the “overtemperature” event but also include data associated the “overtemperature” event, such as one or more temperature measurements and one or more exceeded predetermined thresholds. The RF protocol message is then transmitted by the wireless transceiver 134 of the IMD 100 over the RF communications channel 202 to the charger device 150 using the RF protocol (such as Bluetooth protocol) at 318.

An NF message may also be generated in response to the overtemperature event as well. The NF message may only signal the overtemperature event without the associated data such as one or more temperature measurements and one or more exceeded predetermined thresholds.

In an embodiment, firmware and/or software applications are implemented by one or more processing circuits 126 in the IMD 100 to monitor the temperature measurements and trigger the “exceed heating threshold” event at 310 and/or the “overtemperature” event at 314. In another embodiment, the temperature sensors 136 or other devices may compare the measured temperatures and trigger the overtemperature alert.

In another embodiment, temperature measurements from the one or more temperature sensors 136 in the IMD 100 are monitored at 320 and periodically transmitted in RF protocol messages during charging over the RF communications channel 202 to the charger device 150. The charger device 150 then monitors the temperatures and compares the temperatures to applicable heating thresholds and/or temperature thresholds.

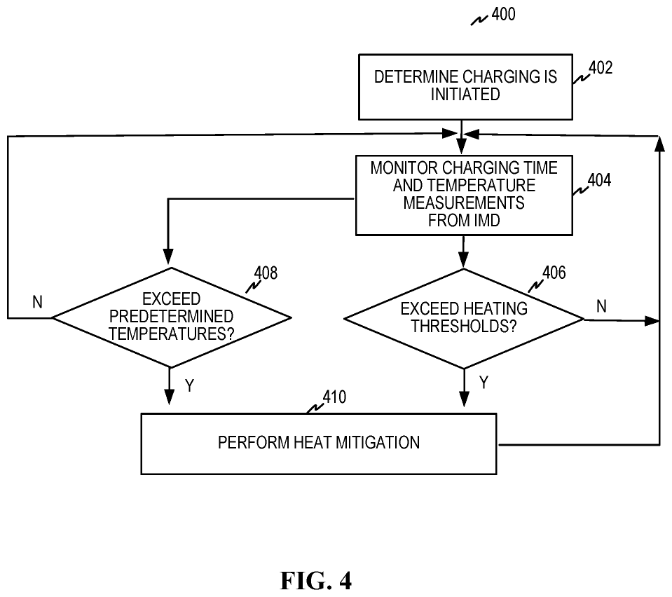

FIG. 4 is a logical flow diagram of an embodiment of a method 400 for monitoring temperatures of an IMD 100 by a charger device 150. In an embodiment, the charger device 150 determines that charging is initiated at 402, e.g. from a clamping message from the IMD 100. In response to the clamping message, the charger device 150 begins to track a time of charging. In addition, the charger device 150 monitors temperature measurements received from the IMD 100 at 404. The temperature measurements are monitored by the IMD 100 and periodically transmitted to the charger device 150 in RF protocol messages over the RF communications channel 202. For example, the temperature measurements may be transmitted by the IMD every second, one minute, five minutes, etc., to the charger device 150.

The charger device 150 compares the temperature measurements and charging time to one or more heating thresholds. When one or more heating thresholds are exceeded at 406, the charger device 150 may then perform heat mitigation at 410. The charger device 150 also compares the temperature measurements to one or more predetermined temperatures. When one or more predetermined temperatures are exceeded at 408, the charger device 150 may then perform heat mitigation at 410.

During wireless charging (e.g. clamping has been detected), the heat mitigation application 166 in the charger device 150 begins to receive and analyze the charging parameters of the charger device 150. For example, the charging parameters may include power output, bridge current, voltage current, etc. The charger device 150 also receives and analyzes temperature data from the IMD 100, e.g. notifications of CEM43 events and/or overtemperature events and/or periodic temperature measurements.

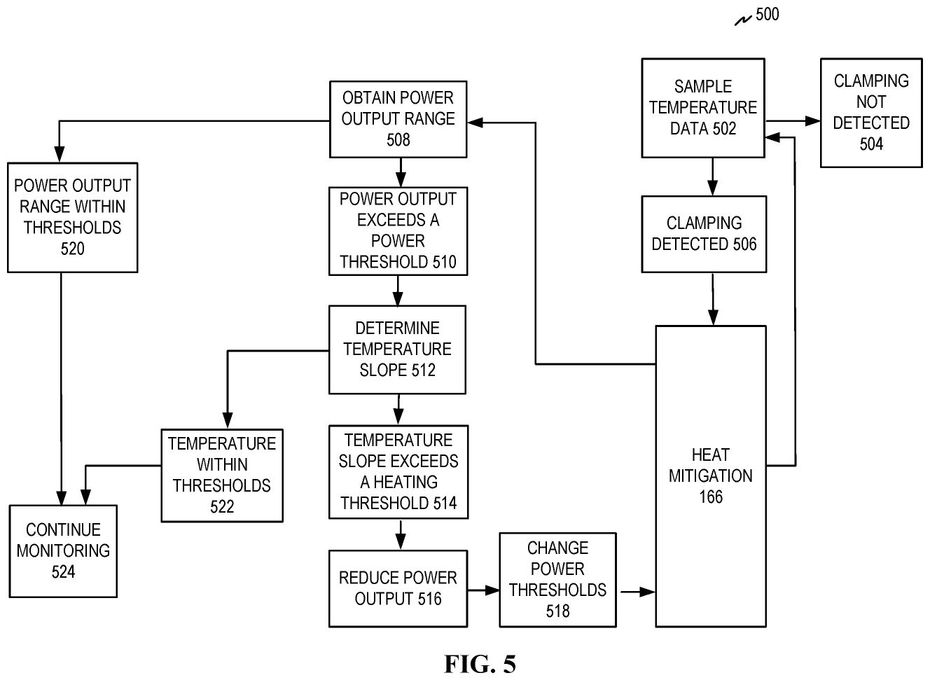

FIG. 5 is a logical flow diagram of an embodiment of a method 500 for heat mitigation of an IMD 100. During wireless charging of the IMD 100, some of the power or energy from the charger device 150 is converted into heat at the charging coil 112 of the IMD 100 and/or at other components of IMD 100. For example, the wireless power or energy from the charger device 150 may be dissipated in the resistive loading presented by the charging coil 112 in the form of heat instead of transformed into electrical current that charges the battery 118. When increased energy levels (e.g., higher power levels) are used to charge the battery 118, the IMD 100 may be charged at a faster rate but the temperature of the IMD 100 may also increase. The charger device 150 may thus control a temperature of the IMD 100 by lowering its output power levels during wireless charging. This lowering of the output power of the charger device 150 may increase the charging time, but this slower charging rate may be preferred by a user to decrease heat and discomfort.

The charger device 150 receives temperature data from the IMD 100 at 502. The temperature data may include periodic temperature measurements and/or notifications of an “exceed heating threshold” event NS “overtemperature” event and/or other data from the IMD 100 associated with its temperature or heating. In addition, the charger device 150 determines whether clamping is detected, e.g. whether charging has been initiated with the IMD 100. If not, the process ends at 504. When clamping is detected at 506, e.g. charging has been initiated with the IMD 100, the heat mitigation application 166 operates to control the charger device 150 to perform one or more functions described herein.

In an embodiment, the charger device 150 evaluates a power output range of its charging module 154 at 508. The power output range of the charger device 150 may be calculated by from the current delivered to the primary coil 152 (the bridge current), the voltage delivered to the primary coil 152 (the bridge voltage) and the phase angle between the bridge current and bridge voltage waveforms. The power output may be determined as follows:

Pₜ=Iₜ*Vₜ*cos θ_{I,V}

wherein Pₜ is the Power Output at time t
Iₜ is the bridge current signal at time t
Vₜ is the bridge voltage signal at time t
θ_{I,V} is the phase angle between I and V waveforms at time t

The power output at time t (Pₜ) may thus be calculated by multiplying the bridge current at time t (Iₜ) by the bridge voltage at time t (Vₜ) and by the cosine of the phase angle between the bridge current and bridge voltage waveforms at time t (cos θ_{I,V}). The power output Pₜ may be sampled at a sampling rate over a time period, such as one to five minutes, to determine a power output range. Other methods may also be used to determine the power output range of the charger device 150.

In this embodiment, the charger device 150 compares the power output range to one or more predetermined power thresholds X. For example, a predetermined power threshold X may initially be set to the operating range of the charger device 150 and the IMD 100. When the power output range is within the one or more predetermined power thresholds at 520, the heat mitigation application 166 continues to monitor at 524. Though the power output range is measured and compared to one or more power thresholds in this example, other charging parameters may additionally or alternatively be determined and compared to one or more other charging thresholds. The one or more other charging parameters include, for example, one or more of: a bridge current, a bridge voltage, or a phase difference between the bridge current and the bridge voltage. Then, one or more other charging thresholds may be predetermined for the respective charging parameter, such as a threshold for the bridge current, threshold for the bridge voltage or a threshold range for the phase difference between the bridge voltage and bridge current.

When the power output range is above one or more power thresholds X at 510, the heat mitigation application 166 further determines whether the temperature measurements from the IMD 100 are over a heating threshold or a predetermined temperature threshold. For example, the charging device may determine a temperature slope (temperature change over time) of the IMD 100 from the received temperature measurements and charging time. The temperature slope may then be compared to a CEM43 heating threshold or another heating threshold. When the determined temperature slope is within the one or more heating thresholds at 522, the heat mitigation application 166 continues to monitor at 524. In addition to the heating threshold, a predetermined temperature threshold may also be set. For example, a predetermined temperature threshold may be set at a maximum temperature (such as 50° C.) that is safe for any amount of charging time. Thus, the temperature measurements may be compared to a heating threshold (e.g., a CEM43 temperature after a charging time) and/or a temperature threshold (a max safe temperature).

When the temperature measurements from the IMD 100 are over a heating threshold or a temperature measurement exceeds a predetermined temperature threshold at 514, the heat mitigation application 166 controls the wireless charger 150 to reduce the power output range at 516. For example, the charger device 150 may decrease the power output range by 10%. The current or voltage delivered to the primary coil 152 of the charger device 150 is then reduced by 10% to lower the power output range by 10%. The applicable power threshold is then similarly reduced by 10% at 518. The charger device 150 may continue to monitor the temperature measurements from the IMD 100. The heat mitigation application 166 may further reduce the power output when the temperature slope continues to exceed the heating threshold Y.

The power output of the charger device 150 may be lowered using the comparison of the measured temperature slopes and the heating thresholds. For example, when the measured temperature slopes exceeds the heating threshold by 5%, the power output may correspondingly be reduced by 5%. In another example, when the measured temperature slopes exceeds the heating threshold by 8%, the power output may correspondingly be reduced by 8%. The charger device 150 may thus determine to decrease the power output by a same percentage that the temperature slope of the IMD 100 exceeds the heating threshold. Similarly, the charger device 150 may determine to decrease the power output by a same percentage that a measured temperature of the IMD 100 exceeds a predetermined temperature threshold. For example, when a measured temperature of 45° C. is 2% over a predetermined temperature threshold of 44° C., the power output is reduced by 2%.

In another embodiment, other correlations may be used to determine an amount to decrease the power output. For example, it may be predetermined that a 1% decrease in power output generates a 5% decrease in a temperature over a time period (temperature slope) of the IMD 100. The patient controller 170 may thus decrease the power output using this correlation and the percentage that the temperature of the IMD 100 exceeds the predetermined temperature threshold.

The reduction in power output is thus selected in response to the percentage that the temperature measurements exceed heating or temperature thresholds. This reduction in power output helps to balance the need to prevent overheating with the need for efficient and timely wireless charging of the IMD 100. The decrease in power output of the charger device 150 reduces the power received by the IMD 100 and slows recharging of the battery 118 to the extent necessary to bring the temperature measurements within thresholds. It is desirable to balance these needs to maintain patient safety and comfort while also providing a manageable charging time for the patient.

In this embodiment, the charger device 150 may not adjust the power output when the one or more charging parameters are not within predetermined charging thresholds at 510, but the temperatures measurements are within heating and temperature thresholds at 522. For example, the charger device 150 may determine to allow an increased power output over power thresholds to charge the IMD 100 when temperature measurements are within applicable thresholds. This embodiment allows for decreased charging times when heating is not a concern for patient safety or comfort.

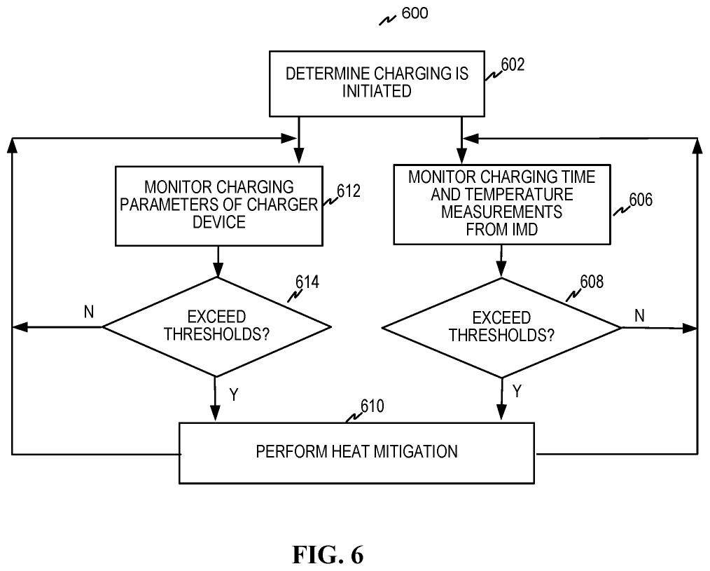

FIG. 6 is a logical flow diagram of another embodiment of a method 600 for heat mitigation of an IMD 100. At 602, the charging of the IMD 100 is initiated. The charger device 150 may receive a clamping signal or other signal from the IMD 100 to indicate initiation of charging. The charger device 150 then begins to track the charging time and monitors temperature measurements of the IMD at 606. When the temperature measurements are within one or more predetermined temperature thresholds or heating thresholds, the heat mitigation application 166 continues to monitor.

When the temperature measurements are not within one or more thresholds, the charger device 150 may initiate one or more heat mitigation processes at 614. For example, the mitigation application 166 may decrease the bridge current to the primary coil 152 to lower a power output to the IMD 100. The charger device 150 may then continue to monitor the temperature measurements from the IMD 100. The heat mitigation application 166 may further reduce the power output if the temperature measurements of the IMD 100 continue to exceed applicable thresholds.

Concurrently, the charging parameters are monitored during wireless charging at 612. The charging parameters may include one or more of power output, a bridge current (e.g., current delivered to the primary coil 152), a bridge voltage (e.g., a voltage delivered to the primary coil 152), a phase value between the bridge current and the bridge voltage or other measurement. When the one or more charging parameters are within predetermined thresholds at 614, the charger device 150 continues to monitor. When the one or more charging parameters are not within predetermined thresholds at 614, the charger device 150 performs heat mitigation at 610. The charger device 150 may determine to adjust the bridge voltage or bridge current to bring the charging parameters within the predetermined charging thresholds. Thus, in this embodiment, the charger device 150 adjusts the power output when either the one or more charging parameters are not within predetermined charging thresholds at 614 or when the temperatures measurements are not within heating or temperature thresholds at 608.

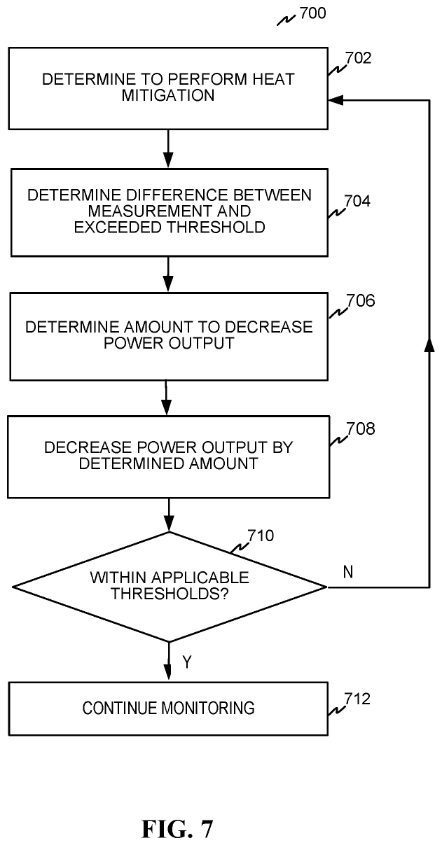

FIG. 7 is a logical flow diagram of another embodiment of a method 700 for determining heat mitigation of an IMD 100. In this embodiment, as described in more detail with respect to FIG. 2 through FIG. 6, one or more of a power threshold, heat threshold and/or temperature threshold may be exceeded. At 702, it is determined to perform heat mitigation, e.g. in response to the one or more exceeded thresholds.

The difference between the exceeded threshold and applicable measurement is determined in 704. The amount or percentage to decrease a power output is determined using this difference at 706. For example, when a temperature measurement after a charging time exceeds a heating threshold by 5%, the power output is reduced by 5% in response thereto. Or when a power measurement exceeds a predetermined power output threshold by 10%, the power output is reduced by 10%.

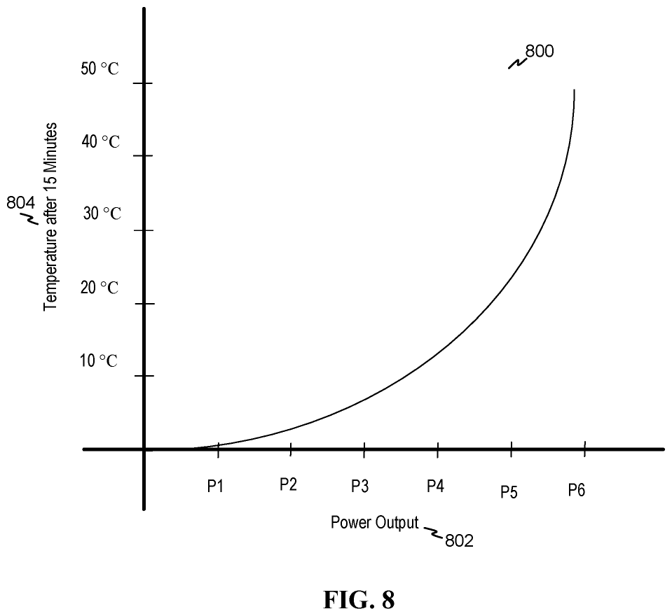

In another embodiment, non-linear correlations of power output to measured heating of the IMD may be used to determine an amount or percentage to decrease power output. FIG. 8 illustrates a graphical representation 800 of a correlation between power output 802 of the charger device 150 and a temperature 804 of an IMD 100 after 15 minutes of charging. The graphical representation 800 is hypothetical based on expected results and not actual experimentation. A correlation is illustrated between a plurality of power outputs 802 (P1-P6) of the charger device 150 and temperature measurements 804 of an IMD 100 after 15 minutes of charging time. This correlation as shown is non-linear. For example, the temperature measurements of the IMD 100 are probably minimal after 15 minutes at a power output of P1. However, the temperature measurements of the IMD 100 increases non-linearly (e.g. exponentially) after 15 minutes at a power output of P5.

So, for example, it may be predetermined through experimentation, that a 1% decrease in power output at power P5 generates a 5% decrease in temperature of the IMD 100 after 15 minutes. As such, in this hypothetical example, when the temperature after 15 minutes of charging is 5% over a heating threshold, the power output P5 is then reduced by 1%.

Through experimentation, the average or mean temperatures after various time periods (5, 10, 15, 30, 60, 90, 120 minutes) may be predetermined for a plurality of power outputs. These predetermined correlations may then be used to determine the percentage to decrease the power output. For example, the predetermined correlations may be used to determine the percentage to lower a power output in order to lower a temperature measurement to within a heating threshold. The decrease in power output may thus be linear or non-linear in response to an amount or percentage that a temperature measurement of the IMD 100 exceeds a heating or temperature threshold.

Referring back to FIG. 7, for heat mitigation, the charger device 150 may then decrease the power output by an amount or percentage that is responsive to the amount or percentage that the temperature measurement of the IMD 100 exceeds a heating threshold at 708. The decrease may be linear or non-linear in relation to the difference that the temperature measurement of the IMD 100 exceeds the heating threshold.

The temperature measurements of the IMD 100 are continued to be monitored at 712 as described in more detail with respect to FIG. 2 through FIG. 6 when heating of the IMD and power of the charger device 150 are within applicable thresholds at 710. When it is determined that one or more applicable thresholds are exceeded at 710, the heat mitigation process may again be performed to decrease the power output of the charger device 150 at 702.

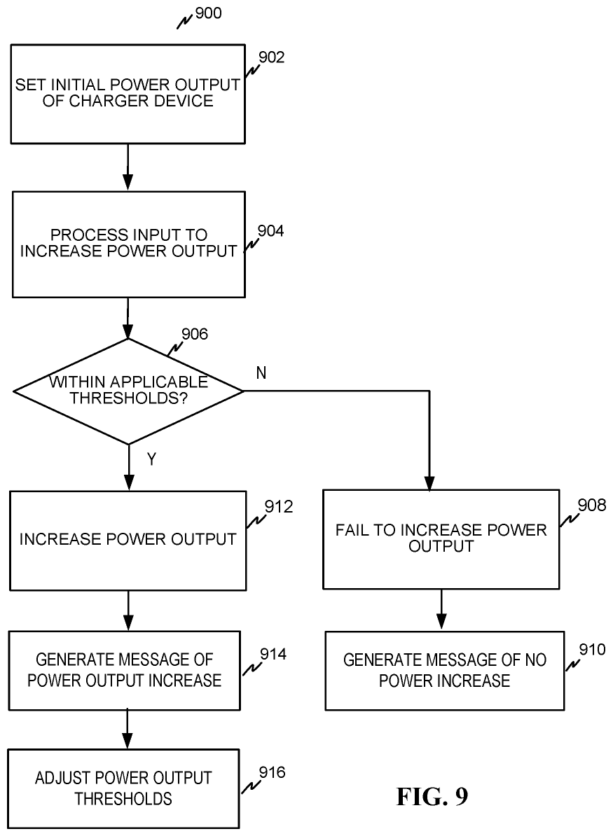

FIG. 9 is a logical flow diagram of an embodiment of a method 900 for modifying power output of a charger device 150. The power output of the charger device 150 may be set at an initial power output at 902, e.g. by a patient, clinician or at manufacture. In an embodiment, the power output of the charger device 150 may be increased in response to a patient input. For example, when a patient is charging an IMD 100 with a charger device 150, the patient may have little to no discomfort and desire to increase power input, e.g. to decrease charging time. The patient or clinician may then input a command or request to increase power output of the charger device 150. The charger device 150 receives this input and processes the request to increase power output at 904.

The charger device 150 then determines whether temperature and power measurements are within applicable thresholds at 906. For example, the charger device 150 determines whether the power output is at a maximum operational power setting for the charger device 150 and/or IMD 100. The charger device 150 may also determine whether other charging parameter (I_{B}, V_{B}) exceed a maximum operational setting. In addition, the charger device 150 determines whether the temperature measurements from the IMD 100 are within heating thresholds. When any applicable thresholds are exceeded at 906, then the charger device 150 fails to increase the power output at 908 and generates a message that indicates no power increase at 910.

When applicable thresholds are not exceeded at 906, then the charger device 150 increases the power output at 912. The increase in power output may include a predetermined increment or a percentage that the power output is under its maximum threshold or other amount. The charger device 150 may then generate a message that indicates a power increase at 914. Any applicable power output thresholds may also be adjusted to the new power output at 916.

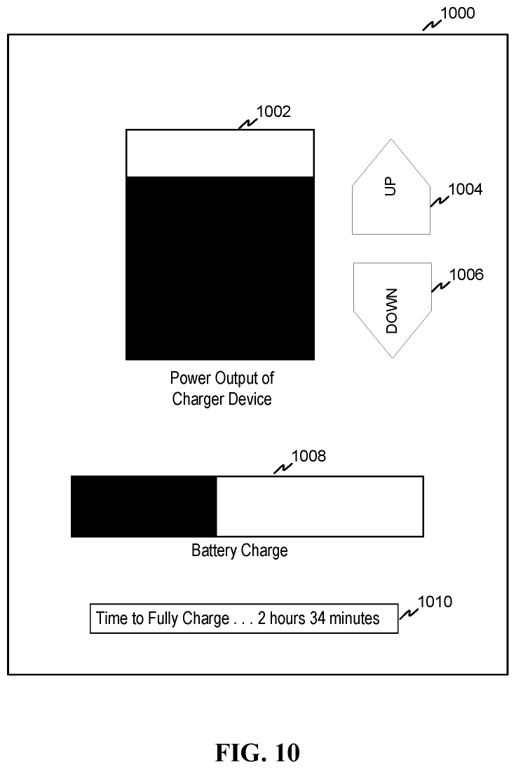

FIG. 10 is a schematic block diagram of an embodiment of a graphical user interface (GUI) 1000 for power management of charger device 150. The GUI 1000 may be generated and displayed by the charger device 150 or the patient controller 170. The GUI 1000 includes a power output display 1002 that indicates a level of the power output of the charger device 150. In this exemplary GUI, the power output display 1002 indicates that the power output level is less than maximum.

The GUI 1000 further includes an input icon 1004 that initiates a request or message to increase a power output of the charger device 150. A patient or clinician may decide to increase the power output using the input icon 1004 on the GUI when the patient is not experiencing discomfort. An increase in power output of the charger device 150 in general will decrease the time to fully charge the battery 118 of the IMD 100. The display 1010 indicates the time to fully charge the battery 118 of the IMD 100. Thus, a patient may see the decrease in this charging time has the power output level is increased and/or as charging progresses. The GUI 1000 may also include a display 1008 that indicates the battery charge. A patient may thus track the progress of the charging of the battery 118 over time.

The GUI 1000 further includes another input icon 1006 that initiates a request or message to decrease a power output of the charger device 150. A patient or clinician may decide to decrease the power output using the input icon 1006 on the GUI when the patient is experiencing discomfort, e.g. from mild heating during charging. A decrease in power output of the charger device 150 in general will increase the time to fully charge the battery 118 of the IMD 100, as indicated in the display 1010. A patient may thus determine the increase in this charging time as the power output level is decreased.

In response to a request for a decrease in power output, the charger device 150 may lower the power output as well as lower one or more heating thresholds. For example, a heating threshold may be dropped by a predetermined amount (such as 0.1° C. to 0.5° C.) upon a patient request. So the heating threshold may be adjusted from a heating threshold of 40° C. after 30 minutes of charging to a new heating threshold of 39.5° C. after 30 minutes of charging.

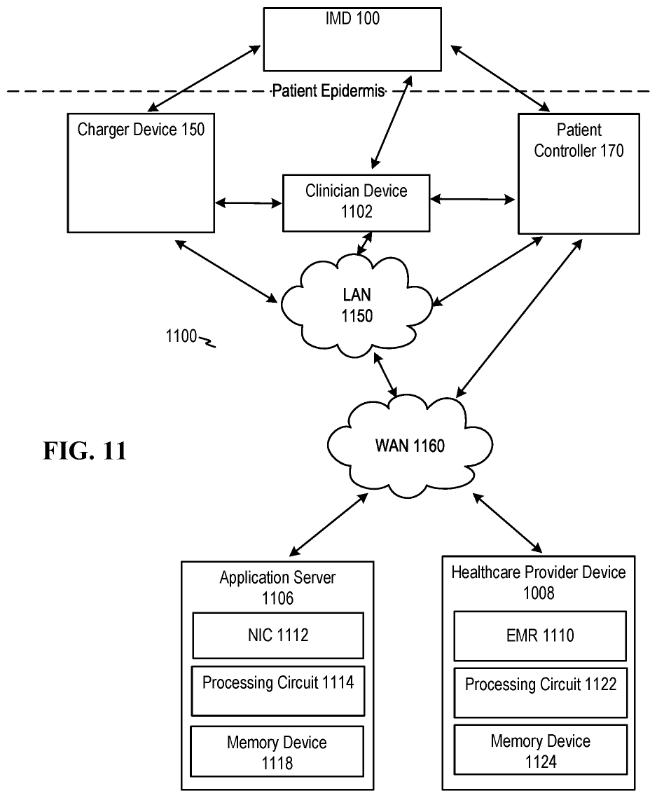

FIG. 11 is a schematic block diagram of an exemplary network 1100 in which the charger device 150 and patient controller 170 may operate. The exemplary network 1100 includes one or more networks that are communicatively coupled, e.g., such as a wide area network (WAN) 1160 and a local area network (LAN) 1150. The WAN 1160 may include a wireless or wired WAN, such as a 4G or 5G cellular network, service provider network, Internet, etc. The LAN 1150 may include a wired or wireless LAN and operate inside a home or enterprise environment. Other networks may be included to communicatively couple the devices, such as edge networks, metropolitan area networks, satellite networks, etc.

The IMD 100 may communicate using a wireless protocol to one or more of the charger device 150 or the patient controller 170 or the clinician device 1102. The patient controller 170 and the charger device 150 may communicate directly using Bluetooth or other wireless or wired protocol or communicate indirectly through the LAN 1150. Though the charger device 150 and the patient controller 170 are shown as separate devices, the charger device 150 may be incorporated into the patient controller 170. The patient controller 170 and/or the charger device 150 may be implemented in a user device, such as a smart phone, laptop, desktop, smart tablet, smart watch, or other electronic device. The clinician device 1102 may be used by a medical professional to program the IMD 100. For example, the clinician device 1102 may set operational modes for neurostimulation as well as set initial power output thresholds, heating thresholds and/or temperature thresholds.

In an embodiment, the charger device 150, patient controller 170 and/or clinician device 1102 may communicate to an application server 1106. The application server 1106 may provide software updates to the charger device 150, the patient controller 170 and/or clinician device 1102. The charger device 150 and/or the patient controller 170 may provide operational data and/or patient data to the application server 1006. The application server 1106 includes a network interface circuit (NIC) 1112 and a server processing circuit 1114. The network interface circuit (NIC) 1112 includes an interface for wireless and/or wired network communications with one or more of the devices in the network 1100. The NIC 1112 may also include authentication capability that provides authentication prior to allowing access to some or all of the resources of the application server 1106. The NIC 1112 may also include firewall, gateway, and proxy server functions. The application server 1106 also includes a processing circuit 1114 and a memory device 1118. For example, the memory device 1118 is a non-transitory, processor readable medium that stores instructions and/or data which when executed or processed by the processing circuit 1114, causes the application server 1106 to perform one or more functions described herein.

In another embodiment, the charger device 150 and/or patient controller 170 may communicate to a local or remote healthcare provider device 1108, e.g. in a physician's office, clinic, or hospital. The healthcare provider device 1108 may store patient or therapeutic information in an electronic medical record (EMR) 1110 associated with the user of the IMD 100. The healthcare provider device 1108 also includes a processing circuit 1122 and a memory device 1124. For example, the memory device 1124 is a non-transitory, processor readable medium that stores instructions and/or data which when executed by the processing circuit 1122, causes the healthcare provider device 1108 to perform one or more functions described herein.

A processing circuit as described herein includes one or more processing devices on one or more printed circuit boards, including one or more of a microprocessor, micro-controller, digital signal processor, video graphics processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. A memory device as described herein includes one or more non-transitory memory devices and may be an internal memory or an external memory to the processing circuit, and the memory device may be a single memory device or a plurality of memory devices. The memory device may be a read-only memory, random access memory, volatile memory, non-volatile memory, static memory, dynamic memory, flash memory, cache memory, and/or any non-transitory memory device that stores digital information.

As may be used herein, the term “operable to” or “configurable to” indicates that an element includes one or more of circuits, instructions, modules, data, input(s), output(s), etc., to perform one or more of the described or necessary corresponding functions and may further include inferred coupling to one or more other items to perform the described or necessary corresponding functions. As may also be used herein, the term(s) “coupled”, “coupled to”, “connected to” and/or “connecting” or “interconnecting” includes direct connection or link between nodes/devices and/or indirect connection between nodes/devices via an intervening item (e.g., an item includes, but is not limited to, a component, an element, a circuit, a module, a node, device, network element, etc.). As may further be used herein, inferred connections (i.e., where one element is connected to another element by inference) includes direct and indirect connection between two items in the same manner as “connected to”.

Note that the aspects of the present disclosure may be described herein as a process that is depicted as a schematic, a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function.

The various features of the disclosure described herein can be implemented in different systems and devices without departing from the disclosure. It should be noted that the foregoing aspects of the disclosure are merely examples and are not to be construed as limiting the disclosure. The description of the aspects of the present disclosure is intended to be illustrative, and not to limit the scope of the claims. As such, the present teachings can be readily applied to other types of apparatuses and many alternatives, modifications, and variations will be apparent to those skilled in the art.

In the foregoing specification, certain representative aspects have been described with reference to specific examples. Various modifications and changes may be made, however, without departing from the scope as set forth in the claims. The specification and figures are illustrative, rather than restrictive, and modifications are intended to be included within the scope of the claims. Accordingly, the scope of the claims should be determined by the claims and their legal equivalents rather than by merely the examples described. For example, the components and/or elements recited in any apparatus claims may be assembled or otherwise operationally configured in a variety of permutations and are accordingly not limited to the specific configuration recited in the claims.

Furthermore, certain benefits, other advantages and solutions to problems have been described above with regard to particular embodiments; however, any benefit, advantage, solution to a problem, or any element that may cause any particular benefit, advantage, or solution to occur or to become more pronounced are not to be construed as critical, required, or essential features or components of any or all the claims.

As used herein, the terms “comprise,” “comprises,” “comprising,” “is comprised of”, “having,” “including,” “includes” or any variation thereof, are intended to reference a nonexclusive inclusion, such that a process, method, article, composition, or apparatus that comprises a list of elements does not include only those elements recited, but may also include other elements not expressly listed or inherent to such process, method, article, composition, or apparatus. Other combinations and/or modifications of the above-described structures, arrangements, applications, proportions, elements, materials, or components used in the practice of the present embodiments, in addition to those not specifically recited, may be varied, or otherwise particularly adapted to specific environments, manufacturing specifications, design parameters, or other operating requirements without departing from the general principles of the same.

Moreover, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is intended to be construed under the provisions of 35 U.S.C. § 112(f) as a “means-plus-function” type element, unless the element is expressly recited using the phrase “means for” or, in the case of a method claim, the element is recited using the phrase “step for.”

1. An external charger device, comprising:
a charging module including at least one primary coil configured to wirelessly transfer power to a charging coil in an implantable medical device (IMD);
a transceiver configured to communicate with the IMD using an RF communications channel and receive one or more temperature measurements from the IMD over the RF communications channel; and
at least one processing circuit and at least one memory device, wherein the at least one memory device stores instructions that, when executed by the at least one processing circuit, causes the external charger device to:
compare the one or more temperature measurements from the IMD to at least one heating threshold; and
determine to perform heat mitigation for the IMD when the one or more temperature measurements exceed the at least one heating threshold.

2. The external charger device of claim 1, wherein the external charger device is configured to perform the heat mitigation for the IMD by adjusting a power output of the charging module.

3. The external charger device of claim 1, wherein the at least one heating threshold includes a predetermined temperature after a predetermined time period of wireless charging.

4. The external charger device of claim 1, wherein the external charger device is configured to:
process an input to lower an operating temperature of the IMD;
decrease a power output of the charging module; and
adjust the at least one heating threshold in response to the input.

5. The external charger device of claim 1, wherein the external charger device is configured to:
monitor a power output range of the charging module; and
compare the power output range to one or more power thresholds.

6. The external charger device of claim 5, wherein the external charger device is configured to:
adjust a power output of the charging module when the power output range exceeds at least one of the power thresholds.

7. The external charger device of claim 5, wherein the external charger device is configured to:
adjust a power output of the charging module when the power output range exceeds the at least one of the power thresholds and when the one or more temperature measurements exceed the at least one temperature threshold.

8. The external charger device of claim 7, wherein the external charger device is further configured to:
monitor a plurality of charging parameters of the charging module, wherein the one or more charging parameters include one or more of: a power output, a bridge current, a bridge voltage, or a phase difference between the bridge current and the bridge voltage; and
compare the plurality of charging parameters to corresponding one or more charging thresholds.

9. An external device, comprising:
a transceiver configured to communicate with an implantable medical device (IMD) using an RF communications channel; and
at least one processing device and at least one memory device, wherein the at least one memory device stores instructions that, when executed by the at least one processing device, causes the external device to:
obtain at least one temperature measurement from the IMD;
determine a temperature slope using the at least one temperature measurement and a charging time;
compare the temperature slope to a heating threshold; and
when the temperature slope exceeds the heating threshold, determine to lower a power output of an external charger device.

10. The external device of claim 9, wherein the heating threshold includes a predetermined temperature after a predetermined time period of wireless charging.

11. The external device of claim 9, wherein the external device is configured to:
process an input originated from a user to lower an operating temperature of the IMD; and
adjust the heating threshold in response to the input.

12. The external device of claim 9, wherein the external device is further configured to:
determine the power output of the external charger device; and
compare the power output to a power threshold.

13. The external device of claim 12, wherein the external device is further configured to:
adjust the power output when the power output exceed the power threshold.

14. The external device of claim 12, wherein the external device is further configured to:
adjust the power output when the power output exceed the power threshold and when the temperature slope exceeds the heating threshold.

15. A method of an external charger device, comprising:
initiating wireless charging of an implantable medical device (IMD);
receiving one or more temperature measurements from the IMD over an RF communications channel;
comparing the one or more temperature measurements from the IMD to at least one heating threshold; and
performing heat mitigation when the one or more temperature measurements exceed the at least one heating threshold.

16. The method of claim 15, further comprising:
adjusting a power output of the external charger device when the one or more temperature measurements exceed the at least one heating threshold.

17. The method of claim 15, wherein the at least one heating threshold includes a predetermined temperature after a predetermined time period of wireless charging.

18. The method of claim 15, further comprising:
receiving a request from a user to lower an operating temperature of the IMD; and
adjusting the at least one heating threshold in response to the request.

19. The method of claim 15, further comprising:
monitoring one or more charging parameters of the external charger device, wherein the one or more charging parameters include one or more of: a bridge current, a bridge voltage, or a phase difference between the bridge current and the bridge voltage; and
comparing the one or more charging parameters to a charging threshold.

20. The method of claim 19, further comprising:
adjusting a power output of the external charger device when the one or more charging parameters exceed the charging threshold.
